(19) United States
(12) Patent Application Publication
(10) Pub. No.: US 2024/0312569 A1
(43) Pub. Date: Sep. 19, 2024

(54) INFORMATION PROCESSING METHOD FOR REPROGRAMMING OF BLOOD CELL

(71) Applicants: CANON KABUSHIKI KAISHA, Tokyo (JP); CANON MEDICAL SYSTEMS CORPORATION, Tochigi (JP)

(72) Inventors: MIE OKANO, Kanagawa (JP); ICHIRO HARADA, Kanagawa (JP); YOJI YAMAMOTO, Tokyo (JP); YASUO SAKURAI, Kyoto (JP)

(21) Appl. No.: 18/605,589

(22) Filed: Mar. 14, 2024

(30) Foreign Application Priority Data
Mar. 15, 2023 (JP) 2023-041312

(51) Int. Cl. G16B 40/10; C12N 5/074; G01N 21/64

(52) U.S. Cl. CPC G16B 40/10; C12N 5/0696; G01N 21/6486

(57) ABSTRACT

Provided is a method of stably reprogramming a cell. Provided is an information processing method for reprogramming of a blood cell, the information processing method including an autofluorescence information acquisition step of acquiring autofluorescence information on a subject blood cell and an analysis step of performing analysis based on the autofluorescence information and cell information on the subject blood cell, in which the cell information includes identification information on the blood cell.

### BACKGROUND OF THE DISCLOSURE

### Field of the Disclosure

The present disclosure relates to an information processing method for reprogramming of a blood cell, an information processing device for executing the information processing method, an information processing program, an electronic medium, a reprogramming device for a blood cell, and a method of manufacturing a pluripotent stem cell.

### Description of the Related Art

The present disclosure relates to fields of stem cells. More specifically, the present disclosure relates to a process of reprogramming a somatic cell, in particular, a blood cell, to produce a pluripotent stem cell.

A pluripotent stem cell, in particular, an induced pluripotent stem cell called “iPS cell,” is established through reprogramming by introduction of a reprogramming factor (for example, Oct4, Klf4, Sox2, c-myc, Lin28, or L-myc) into a somatic cell. However, it is known that efficiency of establishing an iPS cell is low. In addition, stable production is required from the viewpoint of industrialization, and an establishing method (reprogramming method) for an iPS cell continues to be improved. For example, it has been reported that reprogramming efficiency is improved by a histone deacetylase inhibitor, p53 gene suppression, hypoxia culture, and the like, and various studies have still been conducted.

Some cell components are known to emit fluorescence. A plurality of coenzymes relating to a redox state or a metabolic state in a cell emit fluorescence. Fluorescence thus emitted by a substance inherent in a cell is referred to as “autofluorescence.” Research on utilization of autofluorescence for cell sorting has been underway, and in Japanese Patent Application Laid-Open No. 2021-533808, T cells are sorted through use of autofluorescence of cells. Specifically, a fluorescence intensity ratio of coenzymes NADH and FAD possessed by each cell is measured, and a redox state of the cell is quantified, to thereby determine an activation state of each T cell and sort the T cells.

In Japanese Patent Application Laid-Open No. 2010-200676, autofluorescence of cells, E-cadherin expression, and c-kit expression are used to sort pluripotent stem cells from a cell population after reprogramming. However, in Japanese Patent Application Laid-Open No. 2010-200676, while blood cells are sorted, utilization of, for example, an antibody against E-cadherin or c-kit is essential for cell sorting.

### SUMMARY OF THE DISCLOSURE

As described above, there remains a problem of low establishment efficiency in production of pluripotent stem cells. It has also been known that variations in number of colonies and amount of cells that can be acquired occur depending on a manufacturing lot of blood cells or a blood donor. For those reasons, in a case of an unknown cell or donor, a colony obtained after reprogramming cannot be predicted, thereby causing a problem in that, although establishment is successful, the number of colonies obtained is smaller, or conversely larger, than expected. Thus, there has been a demand for a method of stably reprogramming a cell.

In order to solve the above-mentioned problem, the present disclosure provides the following information processing method as one embodiment.

There is provided an information processing method for reprogramming of a blood cell, the information processing method comprising: an autofluorescence information acquisition step of acquiring autofluorescence information on a subject blood cell; and an analysis step of performing analysis based on the autofluorescence information, wherein the analysis step comprises at least any one of: an estimation step of generating an estimation result obtained by estimating a property after reprogramming based on a planned reprogramming condition; or a recommended reprogramming condition generation step of generating a recommended reprogramming condition for the subject blood cell based on a desired property of the cell after reprogramming.

Further features of the present disclosure will become apparent from the following description of exemplary embodiments with reference to the attached drawings.

### BRIEF DESCRIPTION OF THE DRAWINGS

FIG. 1A is a process diagram for illustrating one embodiment of an information processing method.

FIG. 1B is a process diagram for illustrating one embodiment of the information processing method.

FIG. 1C is a process diagram for illustrating one embodiment of the information processing method.

FIG. 2A is a conceptual process diagram for illustrating an example of the information processing method in a case of referring to information about a correspondence relationship.

FIG. 2B is a conceptual process diagram for illustrating an example of the information processing method in the case of referring to the information about the correspondence relationship.

FIG. 2C is a conceptual process diagram for illustrating an example of the information processing method in the case of referring to the information about the correspondence relationship.

FIG. 2D is a conceptual process diagram for illustrating an example of the information processing method in the case of referring to the information about the correspondence relationship.

FIG. 3 is a diagram for illustrating an example of the information about the correspondence relationship.

FIG. 4A is a conceptual process diagram for illustrating an example of the information processing method in a case of using a learning model.

FIG. 4B is a conceptual process diagram for illustrating an example of the information processing method in a case of using the learning model.

FIG. 4C is a conceptual process diagram for illustrating an example of the information processing method in a case of using the learning model.

FIG. 4D is a conceptual process diagram for illustrating an example of the information processing method in a case of using the learning model.

FIG. 5A is a conceptual graph for showing a cell in which a blue component and a green component of autofluorescence are both high in brightness value.

FIG. 5B is an example of a fluorescence brightness distribution chart and a clustering result of cells.

FIG. 6A is an image for showing an example of processing of autofluorescence information on cells.

FIG. 6B is an image for showing an example of the processing of the autofluorescence information on the cells.

FIG. 7A is a process diagram for illustrating an example of a flow of the information processing method.

FIG. 7B is a process diagram for illustrating an example of the flow of the information processing method.

FIG. 7C is a process diagram for illustrating an example of the flow of the information processing method.

FIG. 8 is a diagram for illustrating an example in which the number of colonies after reprogramming has been inferred to acquire a result of the inference.

FIG. 9 is an example of a configuration of an information processing device.

FIG. 10 is examples of an estimation result and a recommended reprogramming condition that are shown on a display unit.

FIG. 11 is experimental data stored in the information processing device in an Example.

FIG. 12A is a presentation screen of an estimation result of the number of colonies or cells after reprogramming in Example 1.

FIG. 12B is a presentation screen of an estimation result of the number of colonies or cells after reprogramming and a recommended reprogramming condition in Examples 1 and 2.

FIG. 12C is a bright-field image of colonies actually obtained through reprogramming under the recommended reprogramming condition in Example 2.

FIG. 13 is a process diagram for illustrating flows of manufacturing of a pluripotent stem cell and corresponding Example numbers.

FIG. 14A is a presentation screen of an estimation result of the number of colonies or cells after reprogramming and a recommended reprogramming condition in Example 3.

FIG. 14B is a bright-field image of colonies actually obtained through reprogramming under a basic reprogramming condition in Example 3.

### DESCRIPTION OF THE EMBODIMENTS

Aspects included in the present disclosure are described below. Embodiments of the present disclosure are described in detail below with reference to the attached drawings. It should be noted that the present disclosure is specified by the claims, and is not construed as being limited to the following modes.

### Information Processing Method

According to this embodiment, there is provided an information processing method for reprogramming of a blood cell, the information processing method including: an autofluorescence information acquisition step of acquiring autofluorescence information on a subject blood cell; and an analysis step of performing analysis based on the autofluorescence information, wherein the analysis step includes at least any one of: an estimation step of generating an estimation result obtained by estimating a property after reprogramming based on a planned reprogramming condition; or a recommended reprogramming condition generation step of generating a recommended reprogramming condition for the subject blood cell based on a desired property of the cell after reprogramming.

The analysis step can include the estimation step of generating an estimation result obtained by estimating a property after reprogramming based on a planned reprogramming condition.

Alternatively, the analysis step can include the recommended reprogramming condition generation step of generating a recommended reprogramming condition for a subject blood cell based on a desired property of the cell after reprogramming. The reprogramming conditions (planned reprogramming condition and recommended reprogramming condition) can include a condition relating to at least any one selected from the group consisting of: information on the number of cells; information on a reprogramming factor; information on a vector amount; information on the number of culture days; and information on a culture vessel.

The analysis step may include both the estimation step and the recommended reprogramming condition generation step.

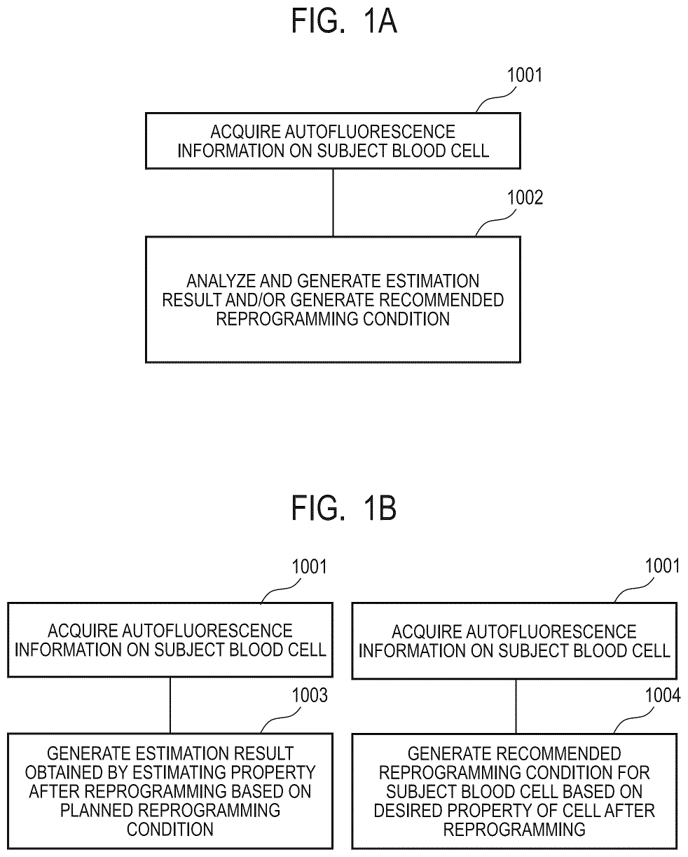

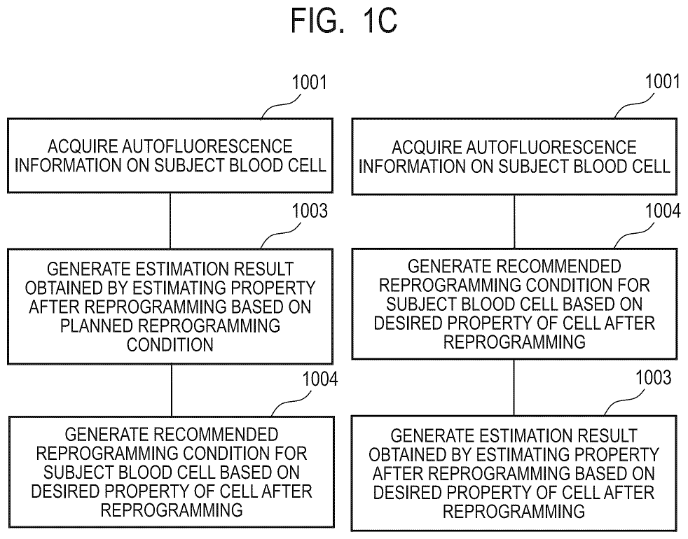

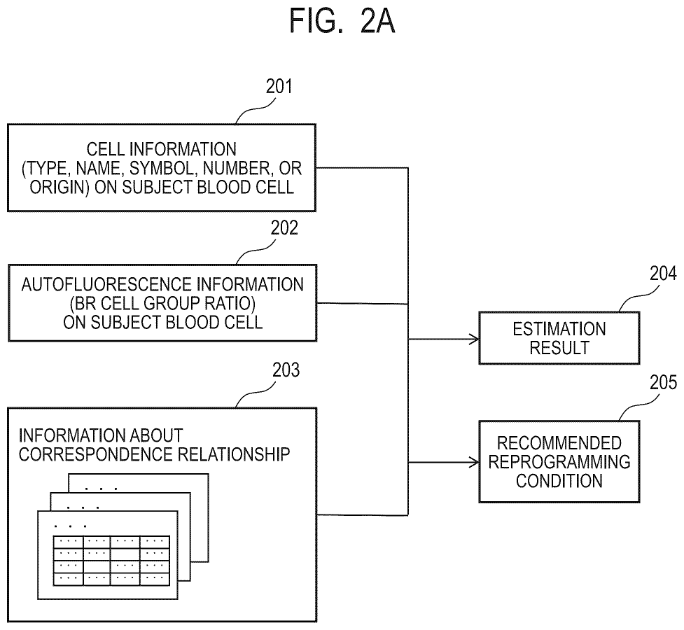

A method according to this embodiment is illustrated in FIG. 1A. In an autofluorescence information acquisition step 1001, autofluorescence information on a subject blood cell is acquired. In an analysis step 1002, analysis is performed based on the autofluorescence information and, as required, cell information on the subject blood cell.

Alternatively, as illustrated in FIG. 1B, the analysis step can include an estimation step 1003 of generating an estimation result obtained by estimating a property after reprogramming based on a planned reprogramming condition.

Alternatively, the analysis step can include a recommended reprogramming condition generation step 1004 of generating a recommended reprogramming condition for a subject blood cell based on a desired property of the cell after reprogramming.

The reprogramming conditions (planned reprogramming condition and recommended reprogramming condition) can include a condition relating to at least any one selected from the group consisting of: information on the number of cells; information on a reprogramming factor; information on a vector amount; information on the number of culture days; and information on a culture vessel.

As illustrated in FIG. 1C, the analysis step 1002 may include both the estimation step 1003 and the recommended reprogramming condition generation step 1004, and in this case, any one of the steps may be executed first. In addition, each step may be included a plurality of times, and an order thereof is not limited in that case.

In a case in which the recommended reprogramming condition generation step 1004 is performed after the estimation step 1003, the recommended reprogramming condition generation step 1004 may be performed after a review of the estimation result generated in the estimation step 1003 or after the reprogramming of the subject blood cell is advanced in consideration of the estimation result. In a case in which the estimation step is performed after the recommended reprogramming condition generation step, the estimation step may be performed after a review of the recommended reprogramming condition generated in the recommended reprogramming condition generation step or further after the reprogramming is advanced in consideration of the recommended reprogramming condition. In such a case, the planned reprogramming condition used to obtain the estimation result may differ from the recommended reprogramming condition in some cases.

In a case in which the analysis step 1002 includes the estimation step 1003 a plurality of times, a second estimation step can be included after a first estimation step when an instruction is further given by a user, first information about the cell after reprogramming is generated based on a first planned reprogramming condition, and second information about the cell after reprogramming is generated based on a second planned reprogramming condition. At this time, the first planned reprogramming condition and the second planned reprogramming condition may be different from each other, and the first information about the cell after reprogramming and the second information about the cell after reprogramming are may also be different from each other.

In a case in which the analysis step 1002 includes the recommended reprogramming condition generation step 1004 a plurality of times, a second recommended reprogramming condition generation step can be included after a first recommended reprogramming condition generation step when an instruction is further given by the user. A recommended first reprogramming condition for the subject blood cell is generated based on a desired property 1 of the cell after reprogramming, and a recommended second reprogramming condition for the subject blood cell is generated based on a desired property 2 of the cell after reprogramming. At this time, the desired property 1 of the cell after reprogramming and the desired property 2 of the cell after reprogramming may be different from each other, and the recommended first reprogramming condition for the subject blood cell and the recommended second reprogramming condition for the subject blood cell may also be different from each other.

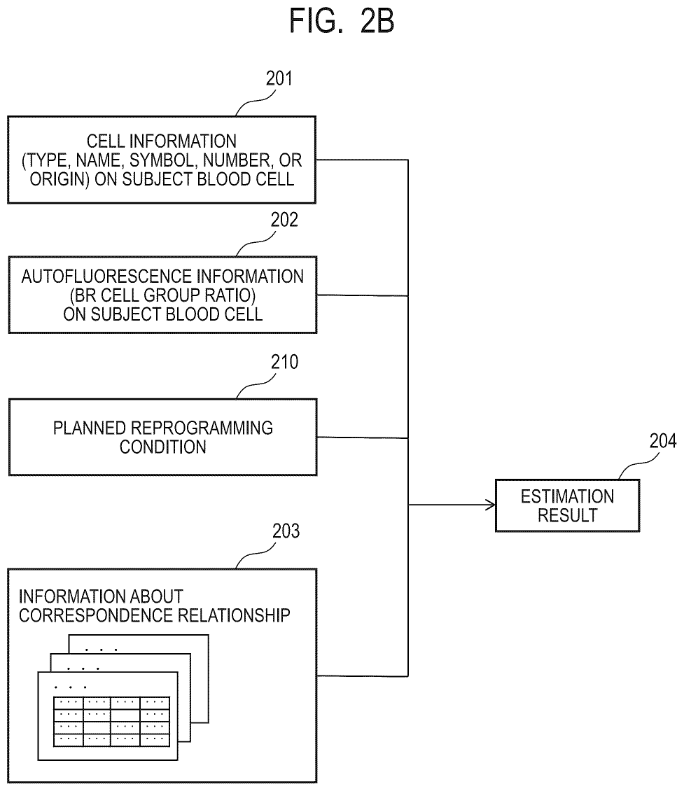

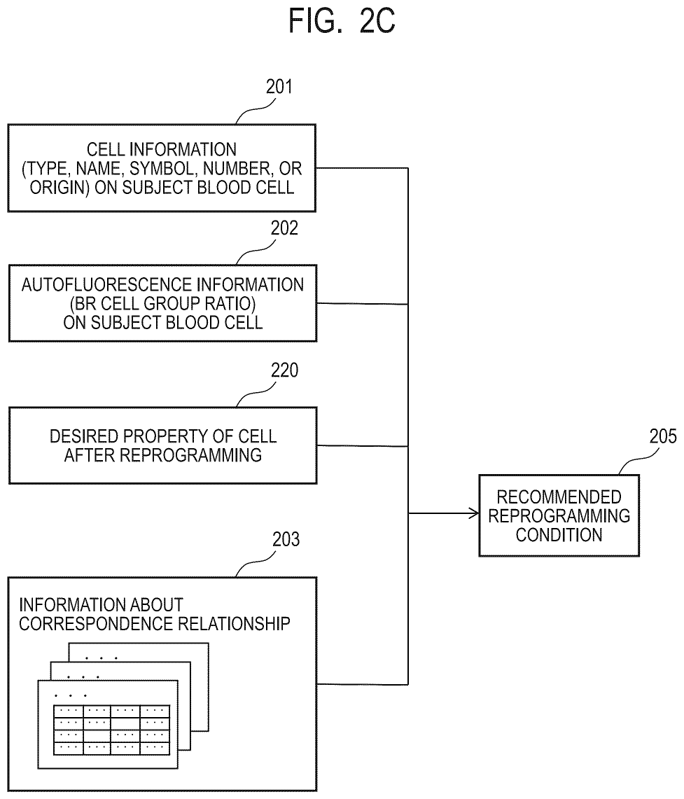

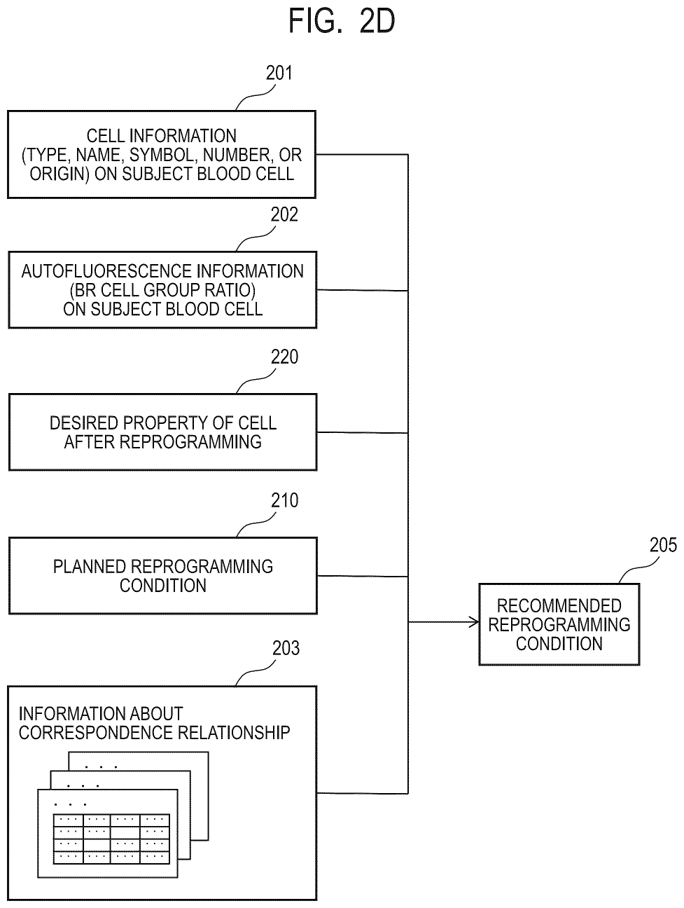

In the analysis step, it is possible to refer to information about a correspondence relationship between the cell information, the autofluorescence information, and the reprogramming condition, and the property after reprogramming (hereinafter sometimes referred to simply as “information about the correspondence relationship”). A conceptual diagram therefor is illustrated in FIG. 2A. Identification information is acquired as cell information 201 on the subject blood cell, and autofluorescence information 202 on the subject blood cell is acquired as well. The identification information refers to information for identifying a subject blood cell, and can include cell information on the subject blood cell, such as a type, a name, a symbol, a number, or an origin thereof. With reference to the cell information 201, the autofluorescence information, the reprogramming condition, and information 203 about the correspondence relationship and also with reference to, for example, data on a cell that has cell information approximate to (for example, data on the same cell type or the same cell line as) that of the subject blood cell, it is possible to generate an estimation result 204 or a recommended reprogramming condition 205.

A case of generating the estimation result 204 is illustrated in FIG. 2B. In the case of generating the estimation result 204, in addition to the autofluorescence information 202 on the subject blood cell and the cell information 201 on the subject blood cell, a planned reprogramming condition 210 that is a reprogramming condition being planned for use in the reprogramming of the subject blood cell is acquired, and with reference to those pieces of information and the information 203 about the correspondence relationship and also with reference to, for example, data using a reprogramming condition approximate to that of the subject blood cell among the data on a cell that has the cell information approximate to that of the subject blood cell, it is possible to generate the estimation result 204. As the estimation result 204, for example, the estimated numbers of cells and colonies can be generated.

A case of generating the recommended reprogramming condition 205 is illustrated in FIG. 2C. In the case of generating the recommended reprogramming condition 205, in addition to the autofluorescence information 202 on the subject blood cell and the cell information 201 on the subject blood cell, a desired property 220 of the cell after reprogramming is acquired, and with reference to those pieces of information and the information 203 about the correspondence relationship and also with reference to, for example, data using a reprogramming condition approximate to that of the subject blood cell among the data on a cell that has the cell information approximate to that of the subject blood cell, it is possible to generate the recommended reprogramming condition 205. In this case, as the recommended reprogramming condition 205, for example, information for use in recommended reprogramming, such as information on the number of cells, information on a reprogramming factor, information on a vector amount, information on the number of culture days, and information on a culture vessel, can be acquired.

The planned reprogramming condition 210 or the desired property 220 of the cell after reprogramming may be input by the user, or may be set in advance. In the case of being set in advance, the planned reprogramming condition 210 or the desired property 220 of the cell after reprogramming is desired to be changeable by the user as required. The desired property of the cell after reprogramming may be the number of cells after reprogramming, a density of colonies after reprogramming, a size of a colony after reprogramming, information about the number of colonies of cells after reprogramming, or the like. Further, information about a rate (ratio) of pluripotent stem cells out of cells obtained after reprogramming may be used. For example, the number of cells or the number of colonies at the fourteenth day after reprogramming may be used.

The autofluorescence information can include information about brightness of a blue component and a green component of autofluorescence. As described later, the information about the brightness of the blue component and the green component of autofluorescence is information about brightness values of the blue component and the green component, and can be set as a ratio of cells (hereinafter sometimes referred to as “BR cells”) in which the brightness values of the blue component and the green component of autofluorescence are both high. The blue component of autofluorescence can be set as fluorescence in a wavelength range of 350 nm or more and 500 nm or less, and the green component of autofluorescence can be set as fluorescence in a wavelength range of 500 nm or more and 600 nm or less.

The reprogramming condition can include a condition relating to at least any one selected from the group consisting of: information on the number of cells; information on a vector amount; information on a reprogramming factor; information on the number of culture days; and information on a culture vessel.

The property after reprogramming can include information about the number of cells after reprogramming.

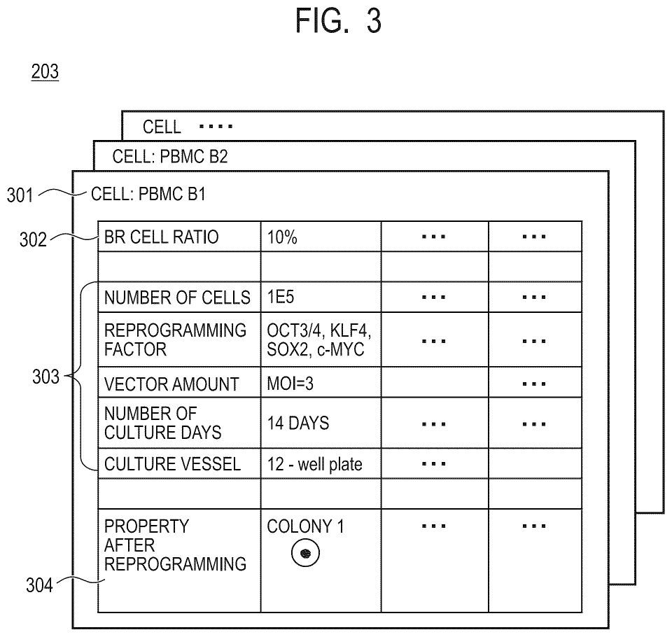

An example of the information about the correspondence relationship between the autofluorescence information and the reprogramming condition, and the property after reprogramming is illustrated in FIG. 3. The information 203 about the correspondence relationship refers to a collection of properties 304 after reprogramming and pieces of autofluorescence information 302 in cases in which cell identification information 301 is variously changed while the reprogramming condition is variously changed. In FIG. 3, an example in which a table is created for each piece of cell identification information 301 such as a cell type and as many thus-created tables as the number of cell types are stacked is illustrated. However, FIG. 3 is a schematic diagram, and may have any format as long as a correspondence between the cell identification information 301, the autofluorescence information 302, and the reprogramming condition 303, and the property 304 after reprogramming is apparent, and the information 203 about the correspondence relationship can be stored so as to enable reference by the information processing system. The correspondence relationship is not required to be entirely shown, but may be partially shown. Experimental data is not always required, and results of simulations may be included.

As illustrated in FIG. 2D, at a time of generating the recommended reprogramming condition 205, the planned reprogramming condition 210 is further acquired in advance, and with reference to the cell information 201 on the subject blood cell, the autofluorescence information 202 on the subject blood cell, the desired property 220 of the cell after reprogramming, the planned reprogramming condition 210, and the information 203 about the correspondence relationship, it is possible to generate the recommended reprogramming condition 205. In this case, the recommended reprogramming condition 205 may be a condition for generating a recommended reprogramming condition that is as close to the input planned reprogramming condition 210 as possible or a condition for proposing a corrected version of the planned reprogramming condition 210. In another case, as the planned reprogramming condition 210 to be first acquired, a condition under consideration (for example, the number of days in a case of reviewing how many days it is suitable to culture) may not be input and only other conditions may be set as the planned reprogramming conditions, while the recommended reprogramming condition 205 to be generated may be limited to a condition under consideration (for example, the number of days that is recommended for culture).

In another case, in the analysis step, it is possible to use a learning model obtained through use of training data in which the cell information, the autofluorescence information, and the reprogramming condition are set as input data and the property after reprogramming is set as output data.

The learning model is a model in which training (learning) has been performed in advance on a machine learning model that conforms to a suitable machine learning algorithm such as deep learning. The learning model having performed learning in advance does not mean that no further learning is to be performed, and may perform additional learning. Additional learning may be performed as required based on data at a time at which the information processing method according to this embodiment is executed.

The learning model may be a regression equation obtained through regression analysis or a neural network model obtained through deep learning or the like, and a data structure, a learning algorithm, and the like of the model are not limited. The learning model may refer to a single neural network in a case in which the learning model is constructed of a neural network and in which an input layer, an intermediate layer, and an output layer are regarded as a unit of a single neural network. Further, in the above-mentioned case, the learning model may refer to a combination of a plurality of neural networks. Further, the learning model may be formed of a combination of a plurality of multiple regression equations, or may be formed of a single multiple regression equation.

The learning to be performed in advance on the machine learning model may be supervised learning using appropriate teacher data (training data), or may be reinforcement learning to be performed so as to maximize reward. In this case, the teacher data refers to training data, and is formed of a pair of input data and output data. The teacher data or the reward may be determined so that a recommended condition for acquiring a desired result is generated.

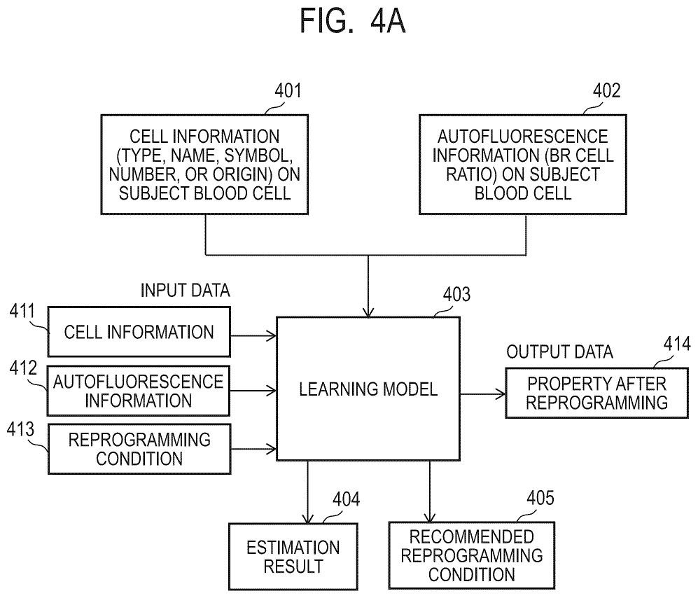

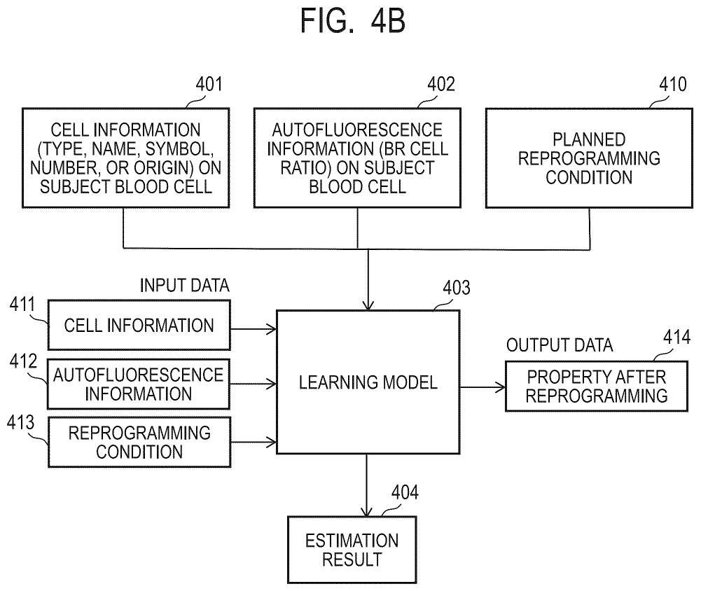

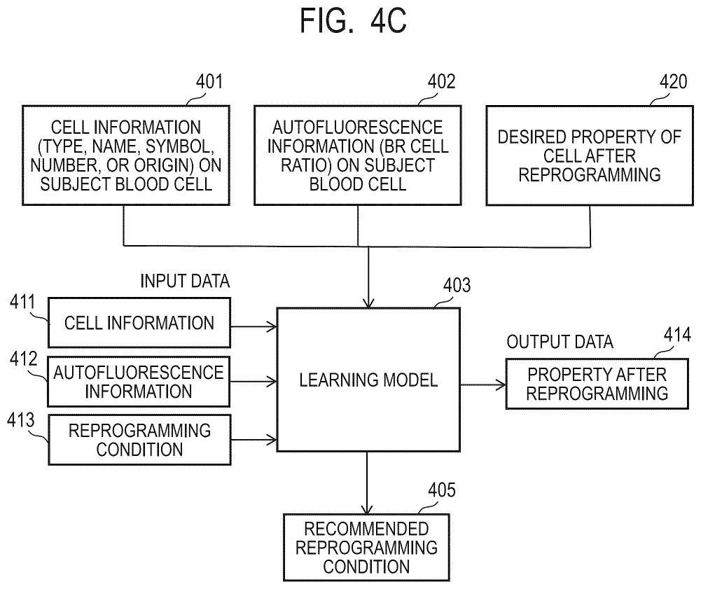

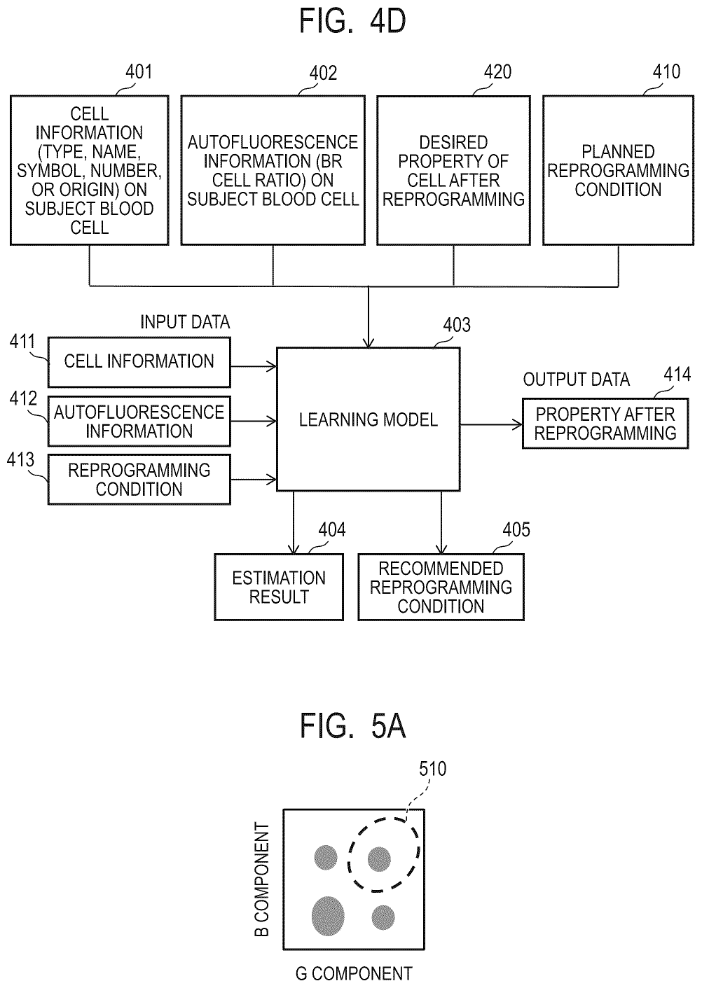

A flow in a case of using the learning model is illustrated in FIG. 4A. As cell information 401 on the subject blood cell, a type, a name, or an origin of the subject blood cell is acquired, and autofluorescence information 402 on the subject blood cell is acquired as well. A learning model 403 can generate, based on those pieces of information, for example, an estimation results 404 or a recommended reprogramming condition 405.

As the learning model 403, it is possible to use the learning model 403 that has performed learning with cell information 411, autofluorescence information 412, and a reprogramming condition 413 being used as the input data and a property 414 after reprogramming being used as the output data.

In a case of generating an estimation result obtained by estimating the property after reprogramming, as illustrated in FIG. 4B, in addition to the autofluorescence information 402 on the subject blood cell and the cell information 401 on the subject blood cell, a planned reprogramming condition 410 that is a reprogramming condition being planned for use in the reprogramming of the subject blood cell is acquired, and the learning model 403 can be used to generate, based on those pieces of information, the estimation result 404. In this case, as the estimation result 404, it is conceivable to generate, for example, the estimated numbers of cells and colonies.

In a case of generating a recommended reprogramming condition for a subject blood cell, as illustrated in FIG. 4C, in addition to the autofluorescence information 402 on the subject blood cell and the cell information 401 on the subject blood cell, a desired property 420 of the cell after reprogramming is acquired, and the learning model 403 can be used to generate, based on those pieces of information, the recommended reprogramming condition 405. In this case, as the recommended reprogramming condition 405, for example, information for use in recommended reprogramming, such as information on the number of cells, information on a reprogramming factor, information on a vector amount, information on the number of culture days, and information on a culture vessel, can be generated. As illustrated in FIG. 4D, at a time of generating the recommended reprogramming condition 405, the planned reprogramming condition 410 of the subject blood cell, which is a reprogramming condition being planned, is further acquired in advance, and the recommended reprogramming condition 405 can be generated through use of the learning model 403 based on the cell information 401 on the subject blood cell, the autofluorescence information 402 on the subject blood cell, the desired property 420 of the cell after reprogramming, and the planned reprogramming condition 410. In this case, the recommended reprogramming condition 405 may be a condition for generating a recommended reprogramming condition that is as close to the input planned reprogramming condition 410 as possible or a condition for proposing a corrected version of the planned reprogramming condition 410. In another case, as the planned reprogramming condition 410 to be first acquired, a condition under consideration (for example, the number of days in a case of reviewing how many days it is suitable to culture) may not be input and only other conditions may be set as the planned reprogramming conditions, while the recommended reprogramming condition 405 to be generated may be limited to a condition under consideration (for example, the number of days that is recommended for culture).

A specific description thereof is given below.

### Blood Cell

The blood cell in this embodiment refers to a cell of blood, and is obtained by differentiating a cell called a hematopoietic stem cell serving as a starting point into a red blood cell, a blood platelet, and a white blood cell. Further, the white blood cell is a generic term for various cells such as neutrophils, eosinophils, basophils, monocytes, and lymphocytes. In this embodiment, a case in which a commercially available human peripheral blood mononuclear cell (PBMC) is used is described, but it is also possible to use a PBMC or a cell obtained through purification from whole blood by a publicly known method, for example, density gradient centrifugation. For example, a product called “Ficoll” or “Vacutainer” can be used to separate a PBMC from blood. A surface antigen or the like may be further used to remove unnecessary cells, and then those cells may be used. In the following, an example in which a PBMC manufactured by Precision for Medicine is used is described, but a product purchased from another manufacturer may be used. Further, a PBMC after separation or a commercially available PBMC may be used as it is, or may be used after pre-culturing or the like is carried out.

Further, the colony in this embodiment refers to a cell aggregate in which cells are not present alone and a plurality of or more cells have gathered. In general, the colony refers to a cell population increased from one cell, but sometimes different cells are mixed therein. Further, the colony of iPS cells has a feature in which the colony has a relatively rounded shape and has an internal structure packed with cells and each cell has a high ratio of a nucleus to a cytoplasm and has the nucleus extending throughout the cell.

### Acquisition of Autofluorescence Information

In acquisition of the autofluorescence information on a cell, for example, an autofluorescence image of cells or the autofluorescence image and a bright-field image of the cells can be acquired. Those images can each be acquired by picking up an image of the cells by an image acquisition device such as a camera through an optical system including a magnifying lens such as a microscope.

The cell may be in any state as long as the autofluorescence information can be acquired, but may be in a live state. For example, in a case of using a general microscope to acquire the autofluorescence image, the cell is may be suspended in and adhered to a Petri dish, a plate, a flask, or the like, and in a case of using flow cytometry or the like, the cells may be dispersed in a solution as a cell suspension. In order to facilitate the acquisition of the autofluorescence information on cell, cells are not in a lump but are in a state in which each of the cells is dispersed or in a state in which each of the cells is recognizable.

The autofluorescence refers to fluorescence emitted by a substance inherent in a cell or a tissue when being irradiated with an excitation wavelength. Examples of an endogenous component that emits autofluorescence include autofluorescent molecules produced in a cell, such as NAD(P)H, flavins (FAD and the like), collagen, fibronectin, tryptophan, and folic acid. It should be noted, however, that in this embodiment, it is not required to identify what endogenous component is emitting autofluorescence, and the autofluorescence may also be fluorescence emitted by a component other than the fluorescent endogenous components exemplified above.

The autofluorescence image is acquired by irradiating a sample including a plurality of cells with one or more kinds of excitation light to acquire one or more pieces of autofluorescence information. The excitation light may be light having one wavelength, or may be light formed of light having a plurality of wavelengths. Further, the fluorescence may be light having one wavelength, or light formed of light having a plurality of wavelengths may be used as the fluorescence.

Further, autofluorescence information can be acquired from an autofluorescence image regarding to the plurality of cells. The autofluorescence information includes brightness of autofluorescence, and may further include position information, area information, and other information on the brightness. The brightness is not particularly limited as long as a value thereof represents a fluorescence intensity, and any index may be used therefor. The brightness may be interpreted as “intensity.”

The brightness may be represented on space coordinates, and may be a value obtained on the coordinates. Color space coordinates are given as an example of the space coordinates. An RGB color space can be used as an example of the color space. The brightness may have a value calculated as an index of at least any one of R, G, or B in the RGB color space.

Brightness information may be described through use of a color space of colors other than those described above.

In this embodiment, two pieces of autofluorescence information, namely, a blue component that is B and a green component that is G, are acquired, but other components may be used. The blue component of autofluorescence may be fluorescence in a wavelength range of 350 nm or more and 500 nm or less, and the green component may be fluorescence in a wavelength range of 500 nm or more and 600 nm or less.

Information about Brightness of Blue Component and Green Component of Autofluorescence

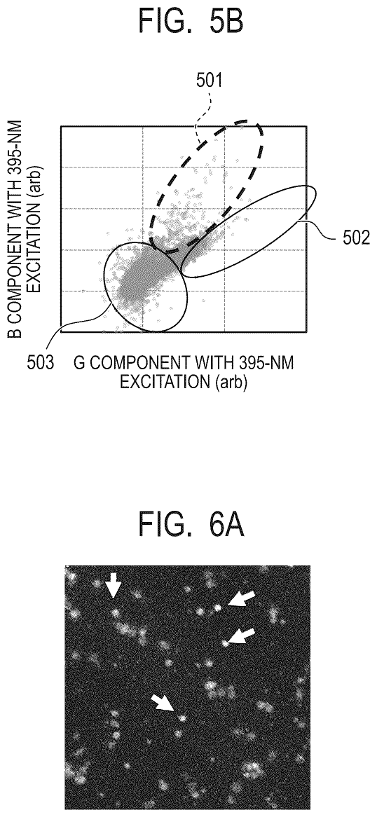

The information about the brightness of the blue component and the green component refers to information about the brightness values of the blue component and the green component. From the information about the brightness values, use of publicly known clustering technology, machine learning, or the like enables classification of a cell group (BR cell group) of cells (BR cells) in which the brightness values of the blue component and the green component of autofluorescence are both high. For example, it is possible to calculate the ratio of BR cells by classifying cells through use of the method and the analysis device as described in Japanese Patent Application Laid-Open No. 2010-200676. A conceptual graph therefor is shown in FIG. 5A. This conceptual graph shows a distribution of cells in a case in which the X-axis indicates the brightness of the green component and the Y-axis indicates the brightness of the blue component. The cell included in a region 510 surrounded by the dotted line, in which the blue component and the green component are greater, is a cell having a high brightness of the G component and a high brightness of the B component, that is, can be set as the BR cell.

FIG. 5B shows an example in which brightness values of blue components and green components were actually acquired for blood cells. This example shows a fluorescence brightness distribution and clustering results of cells in a case in which fluorescence brightness values of green components (G components) are plotted on a horizontal axis and fluorescence brightness values of blue components (B components) are plotted on a vertical axis, the fluorescence brightness values having been obtained when irradiation was performed with excitation light of 395 nm. This is an example of using a publicly known clustering technology, and it is understood from this clustering that the clustering was performed into three groups. That is, the three groups are shown as a region 503 in which the brightness of the blue components and the brightness of the green components are both low, a region 501 (indicated by the dotted line in FIG. 5B) in which the brightness of the blue components and the brightness of the green components are both high and the blue components are relatively greater than the green components, and a region 502 in which the brightness of the blue components and the brightness of the green components are both high but the blue components cannot be said to be relatively greater than the green components.

In such a case, the cells included in the region 501 and the region 502 may be combined to be set as the BR cells. Only cells included in the region 501 can be set as the BR cells.

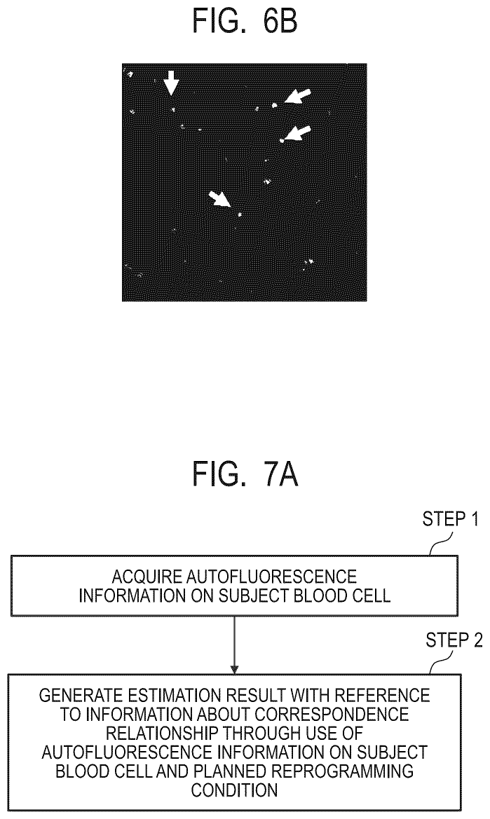

A ratio between the total number of cells and the total number of BR cells may be calculated through use of existing free software. FIG. 6A and FIG. 6B show results of RGB decomposition of an autofluorescence image of blood cells, acquisition of an image of green components, and extraction of cells further having high brightness of the blue component, which were performed through use of ImageJ. FIG. 6A shows the image of green components obtained by the RGB decomposition of the autofluorescence image, and FIG. 6B shows an image obtained after the extraction of the cells further having high brightness of the blue component. In addition, the ratio may be calculated by setting a threshold value of the brightness value and counting the number of BR cells.

It is known that a population having high brightness values of the blue components and the green components of autofluorescence divided as described above includes more cells having a high stemness. The BR cell group is may be a cell group corresponding to the cells having a high stemness indicated by the arrows in FIG. 6A and FIG. 6B, but may include other cells as well. It suffices that a ratio of the cells having a high stemness to all the cells can be relatively determined.

Cells having High Stemness and CD34-Positive Cells

The stem cell refers to a cell that has a differentiation ability to be changed into another cell and a self-renewal ability, and in this specification, a cell that is highly likely to be a stem cell is called “cell having a high stemness.” Examples of the stem cell include a neural stem cell, an epithelial stem cell, a hepatic stem cell, a germ stem cell, a hematopoietic stem cell, and an ES cell, and also include an artificially manufactured stem cell such as an iPS cell. The cell having a high stemness in this embodiment refers to, for example, a CD34-positive cell. The “CD” is one category into which monoclonal antibodies that recognize the same cell membrane antigen or antigenic epitope or the same cell population are grouped, and is also used as a name of a surface antigen recognized by a monoclonal antibody. A CD34-positive fraction is a cell fraction that is rich in human blood stem cells, and has been reported to have a possibility of including a stem cell that differentiates into a cell other than that in a blood system. It has been reported that iPS cells are accordingly established with high efficiency through use of CD34-positive cells of peripheral blood and CD34-positive cells of umbilical cord blood.

### Reprogramming

The reprogramming as used herein means that a nucleus of a differentiated somatic cell is reset to return to a state of a cell nucleus in an early stage of development, such as a fertilized egg, and the differentiated somatic cell is changed to a pluripotent stem cell (hereinafter sometimes referred to as “iPS cell”) or the like, and a condition for performing such reprogramming is set as a reprogramming condition. In regard to the reprogramming condition, there are various parameters such as cells to be used, the number of cells to be used, a vector, a vector amount, a reprogramming factor, the number of culture days, and a culture vessel, and it is known that the numbers of colonies and cells to be obtained vary by changing those parameters. As the cell to be used, for example, the human peripheral blood mononuclear cell (PBMC) can be used, but it is known that a manufacturing lot thereof, a donor thereof, the number of cells to be used, a cell concentration, a fluid amount, and the like affect the number of colonies or cells after reprogramming. Similarly in regard to the vector, various vectors are offered for sale, and the type or amount of vector to be used affects a result. Known reprogramming factors include the Oct family (for example, Oct3/4), the Sox family (for example, Sox2, Sox1, Sox3, Sox15, and Sox17), the Klf family (for example, Klf4 and Klf2), the myc family (for example, c-myc, N-myc, and L-myc), Nanog, and Lin28. In regard to an iPS establishing method, a large number of documents have been published, and reprogramming conditions and reprogramming protocols that serve as a guide have been described by manufacturers of vectors, and hence such conditions and protocols are usable as a reference.

In the following example, a result of using an iPS-cell induction protocol from human peripheral blood mononuclear cells and monocytes using SRV iPS-2 Vector sold by TOKIWA-Bio Inc. is given as an example, but the vector and the protocol are not limited thereto. A vector manufactured by another manufacturer or a vector that is not commercially available may be used, and any protocol can be used.

In the following example, a basic reprogramming condition was set as follows. A vector was added to a pellet of 1E5 cells so that MOI=3, and such an amount of a culture medium for blood cells as to have a volume of 20 μL to 33 μL in combination with the vector was added, to thereby cause the cells and the vector to react with each other. After the reaction, reaction and washing were carried out in accordance with a protocol recommended by the manufacturer, and then 0.75 mL of seeding medium was added to a 12-well plate coated with iMatrix-511. After that, culture medium exchange was carried out in accordance with the protocol, and culture was performed for 14 days. In the following example, iMatrix-511 was prepared so as to have a concentration of 0.5 μg/cm², and 0.75 mL thereof was coated on the 12-well plate.

The MOI means multiplicity of infection, and represents a ratio of infectious viruses to cells to be infected thereby.

### Analysis

A specific example of the analysis is given below. In this specific example, the cell information on the subject blood cell, the autofluorescence information (BR cell group ratio) on the subject blood cell, and the planned reprogramming condition were acquired, and an estimation result was generated with reference to the information about the correspondence relationship. That is, this specific example is an example of the embodiment illustrated in FIG. 2B.

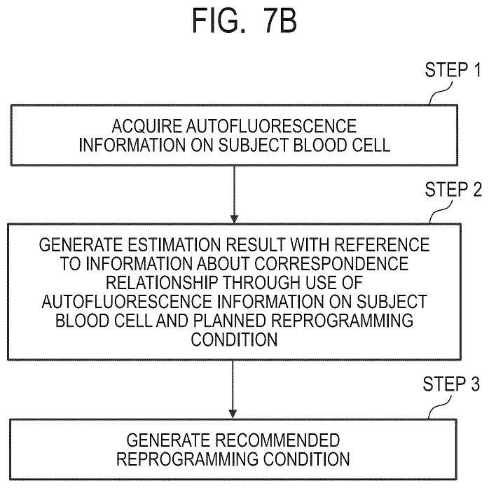

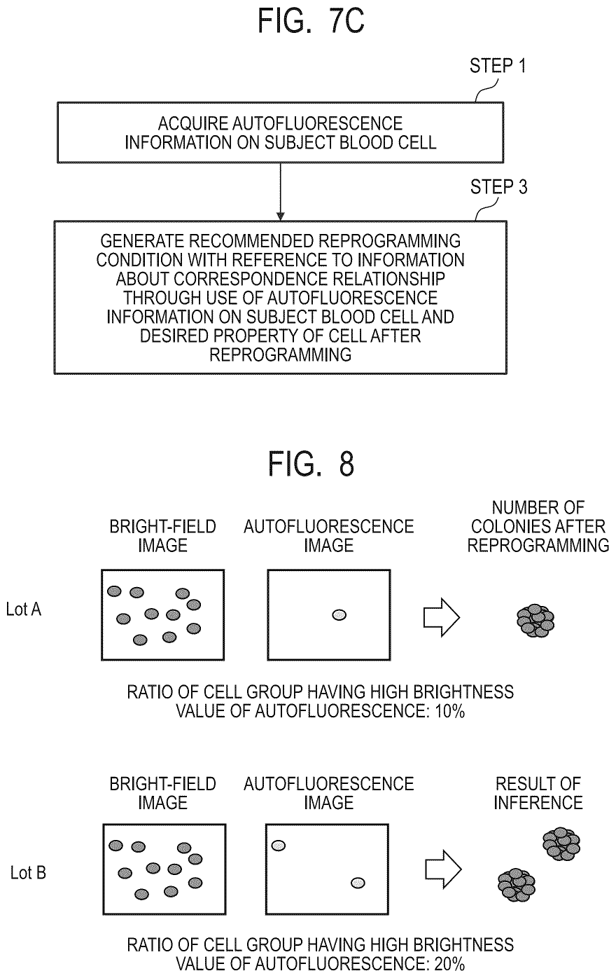

A flow thereof is as illustrated in FIG. 7A. An experiment result obtained when a reprogramming factor was introduced to a lot A of commercially available PBMCs under the above-mentioned basic reprogramming condition is used as the information about the correspondence relationship to estimate the number of colonies obtained when a lot B serving as the subject blood cell is reprogrammed under the basic reprogramming condition. First, in Step 1, the autofluorescence information is acquired. Specifically, a cell suspension of the lot B is prepared and dropped onto a slide glass to acquire the bright-field image and the autofluorescence image. Two pieces of autofluorescence information, namely, the blue component that is B and the green component that is G, are acquired from the autofluorescence image.

Subsequently, in Step 2, the cell information, the autofluorescence information, and the reprogramming condition are used to estimate the number of colonies after reprogramming and generate an estimation result. As illustrated in a conceptual diagram of FIG. 8, the total number of cells is calculated from the bright-field image, and the number of cells of a cell group having high brightness values of the blue component and the green component, that is, the BR cell group, is counted from the autofluorescence image, to thereby calculate a ratio of the BR cell group to the total number of cells. In calculation of the total number of cells and the number of cells of the BR cell group, a method described in the information about the brightness of the blue component and the green component of autofluorescence can be used to perform the calculation. In this example, in regard to the lot A, the ratio of the BR cell group and a result of reprogramming are known. The lot A and the lot B are approximate to each other in terms of cell information, and hence the description is given by assuming a case in which a result of the lot A is set as reference information selected from the information about the correspondence relationship to carry out information processing through use of the result of the lot A. It is assumed that, in the lot A, the ratio of the BR cell group was 10% and the number of obtained colonies was 1 as a result of carrying out reprogramming under the above-mentioned basic reprogramming condition. In a case in which the ratio of the BR cell group in the lot B is 20%, when the lot B is reprogrammed under the same condition as that of the lot A, the number of colonies after reprogramming can be estimated to be 2 from (number of colonies after reprogramming in the lot A)×[(ratio (20%) of the BR cell group in the lot B)/(ratio (10%) of the cell group having a high brightness value of the lot A)].

Although the number of colonies has been estimated in this case, the colony of the lot A may be peeled off, the number of cells may be counted after suspension, and the number of cells after reprogramming of the lot B may be estimated from a value of the counting. Further, how many cells a population formed by one colony has may be calculated in advance from the number of culture days and the size of the colony, and the estimation may be performed through use of a value of the calculation. In addition, an area occupied by a colony or a cell may be calculated, to thereby perform the estimation. The estimation result may be an image diagram of a colony obtained after reprogramming in addition to the number of colonies and the number of cells.

In the above-mentioned example, the number of colonies obtained in the case of using the lot B has been estimated on the assumption that the ratio of the cell group having a high autofluorescence brightness value and the number of colonies after reprogramming are in a linear relationship, but a method for the estimation is not limited thereto.

For example, a relationship between the autofluorescence information and the number of colonies or cells after reprogramming may be acquired in advance through use of a plurality of lots, and the estimation can also be performed from a result of a lot close to the autofluorescence information on the lot B to be estimated.

In the above-mentioned example, the information about the correspondence relationship is the number of colonies (property after reprogramming) at a time at which the lot A (cell information) of the commercially available PBMCs was reprogrammed under the above-mentioned basic condition (reprogramming condition), while subject cells are the lot B (cell information). The above-mentioned example has shown that the estimation result (number of colonies) has been obtained when the subject cells are reprogrammed under the above-mentioned basic condition (planned reprogramming condition).

As described above, according to this embodiment, the estimation result after reprogramming can be obtained, and the user can determine, from the estimation result, whether it is required to use the current reprogramming condition for the establishment or to change the reprogramming condition.

As illustrated in FIG. 7B, after the estimation result is generated in Step 2, a recommended reprogramming condition may be further generated as described in Step 3. In this example, the recommended reprogramming condition can be set to a condition obtained by changing one of or one or more of the number of cells to be used for reprogramming, the vector amount, the reprogramming factor, the number of culture days, and the culture vessel among the parameters of the basic reprogramming condition. In this example, when 10 colonies are desired to be obtained, it is possible to recommend, for example, a condition of setting a pellet of 5E5 cells as the cells to be used for reprogramming. In another case, when the reprogramming is carried out with the number of cells being kept under the same condition, a condition of increasing the number of culture days may be recommended.

As described above, according to this embodiment, it is possible to estimate information about the number of colonies or cells after reprogramming from the autofluorescence information and the reprogramming condition, generate an estimation result, and generate a recommended reprogramming condition. In addition, the user can stably manufacture an iPS cell by carrying out reprogramming through use of the recommended reprogramming condition obtained in the above-mentioned manner.

In the above-mentioned example, the information about the number of colonies or cells after reprogramming is estimated through use of the cell information, the autofluorescence information on the subject blood cell, and the desired property of the cell after reprogramming, an estimation result is generated, and a recommended reprogramming condition is generated. However, the autofluorescence information may be acquired, and a recommended reprogramming condition may be generated from the autofluorescence information. That is, as illustrated in FIG. 7C, the processing step of Step 2 may be omitted. It is possible to select a cell that has approximate cell information from the information about the correspondence relationship, find out a result satisfying the reprogramming condition satisfying the desired property of the cell after reprogramming from the autofluorescence information, the reprogramming condition, and the property after reprogramming, and set the obtained result as the recommended reprogramming condition.

Further, as described above, a learning model obtained by a method of machine learning or the like may be used in place of the information about the correspondence relationship to be referred to.

Information Processing Device, Information Processing Program, and Electronic Medium having Information Processing Program stored thereon.

As a further embodiment, the present disclosure provides an information processing device that executes the information processing method according to the above-mentioned embodiment. The information processing device acquires autofluorescence information on a subject blood cell, and performs analysis based on the autofluorescence information and cell information on the subject blood cell.

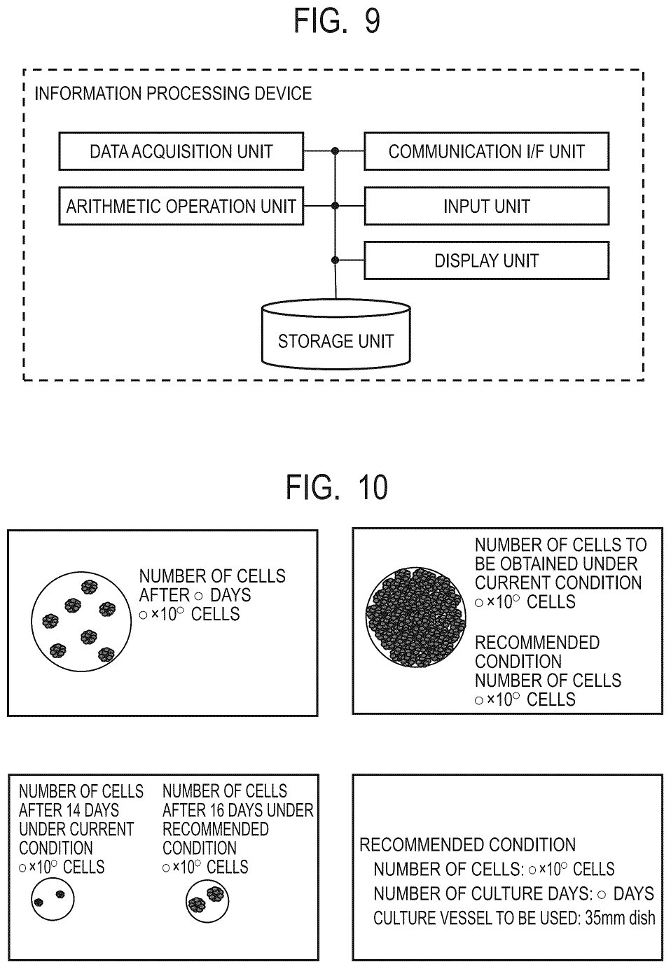

FIG. 9 is an example of a configuration of the information processing device according to this embodiment. The information processing device has functions of a computer. For example, the information processing device may be formed integrally with a desktop personal computer (PC), a laptop PC, a tablet PC, a smartphone, or the like.

The information processing device that performs an arithmetic operation and storage includes a storage unit, a data acquisition unit, an arithmetic operation unit, a communication interface (I/F) unit, a display unit, and an input unit in order to implement functions as a computer that performs an arithmetic operation and storage. In FIG. 9, the information processing device formed of the respective units is illustrated as an integral device, but a part of those functions may be externally provided. For example, the display unit and the input unit may be externally provided.

The storage unit is, for example, a random access memory (RAM), a read only memory (ROM), or a hard disk drive (HDD). The autofluorescence information, the estimation result, the reprogramming condition, and the like can be recorded in this storage unit.

The data acquisition unit has a function of acquiring the autofluorescence information. For example, a device such as a microscope, a camera, or a device for flow cytometry may be incorporated into the information processing device as the data acquisition unit. Further, the data acquisition unit may not be incorporated into a device main body, and the data may be acquired from an external device through a communication interface (I/F) or the storage unit.

The arithmetic operation unit is, for example, a CPU, and has a function of performing a predetermined operation in accordance with a program stored in the RAM, the HDD, or the like, and controlling each unit of the information processing device. The arithmetic operation unit further receives data from the storage unit, the input unit, the communication I/F unit, and the like, and performs control and an arithmetic operation on the computer. The arithmetic operation unit has a function of actually performing the information processing method according to the above-mentioned embodiment to calculate the estimation result and the recommended reprogramming condition. The arithmetic operation unit is also responsible for sending a result of the arithmetic operation to the display unit.

The communication I/F unit is a communication interface based on standards such as Wi-Fi (trademark) and 4G, and is a module for communicating to/from another device. For example, autofluorescence information acquired by an image acquisition device such as a microscope can be stored in the storage unit through Wi-Fi.

The display unit is a liquid crystal display, an organic light emitting diode (OLED) display, or the like, and is used for display of a moving image, a still image, characters, and the like. The display unit has a function of displaying, as illustrated in FIG. 10, the estimation result, the recommended reprogramming condition, and the like that have been calculated by the arithmetic operation unit.

The input unit is a button, a touch panel, a keyboard, a pointing device, or the like, and is used by a user to operate the information processing device. The input unit can also be used to input the reprogramming condition, the number of colonies or cells desired after reprogramming, and the like. The display unit and the input unit may be integrally formed as a touch panel.

The configuration of devices illustrated in FIG. 9 is an example, and a device other than those may be added, or some of the devices may not be provided. Further, some of the devices may be replaced by other devices having the same functions. Further, some of the functions may be provided by another device through a network, or the functions that form this embodiment may be implemented by being distributed to a plurality of devices. For example, the HDD may be replaced by a solid state drive (SSD) that uses a semiconductor device such as a flash memory, or may be replaced by a cloud storage.

An example in which the above-mentioned device is used to estimate the autofluorescence information on the subject blood cell and the property after reprogramming from the planned reprogramming condition is illustrated. The arithmetic operation unit estimates the property after reprogramming from the autofluorescence information on the subject blood cell stored in the storage unit through the communication I/F unit and the planned reprogramming condition newly input by the user through the input unit. As the reprogramming condition, a condition that has been input in advance to the HDD may be used. Further, the estimation may be performed through use of the information about the correspondence relationship formed of existing experiment results stored in the storage unit or through use of a learning model. The estimated result can be displayed on the display unit. As illustrated in FIG. 10, the display unit may display any one of the generated estimation result or the recommended reprogramming condition, or may display both the estimation result and the recommended reprogramming condition. As the estimation result, the number of colonies or cells acquired on a day on which culture has been performed for the number of culture days set in the planned reprogramming condition may be displayed, or an image diagram of the cells at that time may be displayed. Further, both the number of cells or colonies obtained in a case of employing the planned reprogramming condition and the number of cells or colonies obtained under the recommended reprogramming condition may be displayed.

A further embodiment provides an information processing program for causing an information processing device to execute the information processing method according to the above-mentioned embodiment. The information processing program can be handled as the information processing device by being built into the computer or the like.

A further embodiment provides an electronic medium having stored thereon the above-mentioned information processing program. Examples thereof include a magnetic tape, a floppy disk, an optical disk, a memory card, an HDD, an SSD, and a USB flash drive. The electronic medium refers to a medium that can physically record information and is subjected to recording and readout due to electromagnetic actions. The information processing device can also be formed by installing a program in an existing computer through this electronic medium.

### Reprogramming Device for Blood Cell

The present disclosure provides, as a further embodiment, a reprogramming device for a blood cell, the reprogramming device including: an image pickup unit configured to acquire autofluorescence information on a subject blood cell; an information processing system; and a reprogramming factor introduction unit configured to introduce a reprogramming factor into the subject blood cell, wherein the information processing system includes: an autofluorescence information acquisition unit configured to acquire the autofluorescence information from the image pickup unit; and an analysis unit configured to perform analysis based on the autofluorescence information, and wherein the analysis unit includes at least any one of: an estimation unit configured to generate an estimation result obtained by estimating a property after reprogramming based on a planned reprogramming condition; or a recommended reprogramming condition generation unit configured to generate a recommended reprogramming condition for the subject blood cell based on a desired property of the cell after reprogramming.

Specifically, the reprogramming device includes the information processing device according to the above-mentioned embodiment, an image pickup unit for acquiring autofluorescence information to be provided to the information processing device, and a reprogramming factor introduction unit that introduces a reprogramming factor into a subject blood cell.

The analysis unit of the information processing system includes the recommended reprogramming condition generation unit configured to generate the recommended reprogramming condition for the subject blood cell, and the reprogramming factor introduction unit is configured to introduce the reprogramming factor into the subject blood cell in accordance with the recommended reprogramming condition.

The image pickup unit is only required to be able to acquire an autofluorescence image of cells, and may also be able to acquire a bright-field image thereof. The image pickup unit can include excitation light, a lens, a detector, and the like, and a fluorescence microscope may be used as the image pickup unit.

The reprogramming factor introduction unit is only required to be able to perform the above-mentioned reprogramming processing, and can include a robot arm, a pump, a pipette, a moving belt, an incubator, and the like.

### Method of Manufacturing Pluripotent Stem Cell

The present disclosure provides, as a further embodiment, a method of manufacturing a pluripotent stem cell, the method including: an autofluorescence information acquisition step of acquiring autofluorescence information on a subject blood cell; an analysis step of performing analysis based on the autofluorescence information; and a step of reprogramming the subject blood cell, wherein the analysis step includes at least any one of: an estimation step of generating an estimation result obtained by estimating a property after reprogramming based on a planned reprogramming condition; or a recommended reprogramming condition generation step of generating a recommended reprogramming condition for the subject blood cell based on a desired property of the cell after reprogramming.

The analysis step can include the recommended reprogramming condition generation step of generating the recommended reprogramming condition for the subject blood cell, and the reprogramming factor introduction unit can reprogram the subject blood cell in accordance with the recommended reprogramming condition.

Further, the analysis step can further include, after a recommended reprogramming condition generation step of generating a recommended first reprogramming condition for the subject blood cell based on a desired property 1 of the cell after reprogramming, when there is a request of a user, a recommended reprogramming condition generation step of generating a recommended second reprogramming condition for the subject blood cell based on a desired property 2 of the cell after reprogramming. The subject blood cell can be reprogrammed in accordance with the recommended second reprogramming condition.

### EXAMPLES

A more specific description is given below with reference to Examples. The embodiment of the present disclosure is not limited to the following Examples.

### Example 1

In this Example, PBMCs obtained by pre-culturing commercially available PBMCs for 5 days were used. The pre-culturing means growing stem or progenitor cells among mononuclear cells through culturing in a culture medium to which a cytokine is added. In regard to a protocol for pre-culturing, the iPS cell establishment protocol ver. 1.1 for research published by CiRAF was used to carry out pre-culturing. The PBMCs obtained as described above were used as blood cells. The obtained cells are hereinafter referred to as “PBMC lot C” or simply as “lot C.” In this Example, the lot C was set as subject blood cells. Now, description is given in accordance with a flow of FIG. 7B.

First, in Step 1, the autofluorescence information on the lot C was acquired. A cell suspension of the PBMC lot C was prepared and dropped onto the slide glass to acquire the bright-field image and the autofluorescence image. The method using ImageJ described above was used to calculate a ratio of a population in which the brightness values of the blue component and the green component are both high. As a result, the ratio of the population having high brightness values of the blue component and the green component of the PBMC lot C was 10%.

Subsequently, in Step 2, an estimation result obtained by estimating the number of colonies after reprogramming was generated through use of the autofluorescence information on the lot C and the planned reprogramming condition. As the planned reprogramming condition, a condition for using SRV iPS-2 Vector of TOKIWA-Bio was used. Information processing was carried out by setting the number of cells to be used for reprogramming to 1E5 cells (with a fluid amount of 10 μL), the vector amount to such an amount as to satisfy MOI-3, the culture vessel to be used to a 12-well plate, and the culture period to 14 days. Further, it was set as the desired property of the cell after reprogramming that each of colonies was to be observable and a required amount of iPS cells was to be obtained.

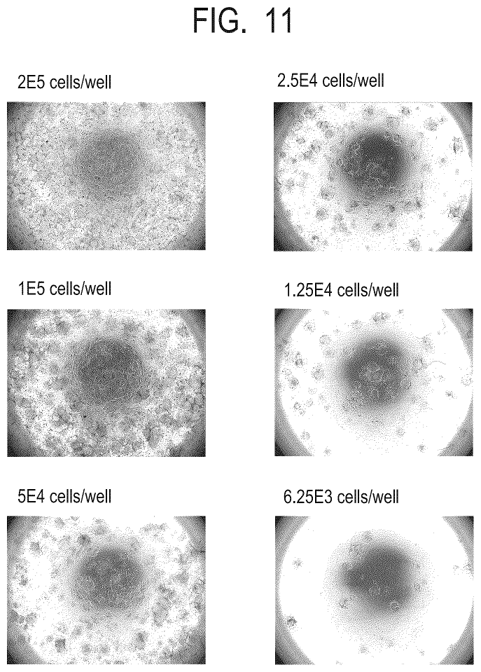

In this Example, a plurality of pieces of experimental data acquired in advance with the PBMCs being used as reference cells were stored in the HDD. The experimental data included the number of cells used for reprogramming and the number of colonies obtained after reprogramming. FIG. 11 shows images of colonies after reprogramming at a time of using the PBMCs 10% of which was the population having high brightness values of the blue component and the green component among the reference cells.

In this manner, the ratio of the population having high brightness values of the blue component and the green component, the number of cells used for reprogramming, and the number and images of colonies that were obtained are stored in advance in the device, to thereby be able to generate an estimation result and a recommended reprogramming condition. Although colony images were stored in this Example, numerical information thereon may be stored in place of the images, and may be used.

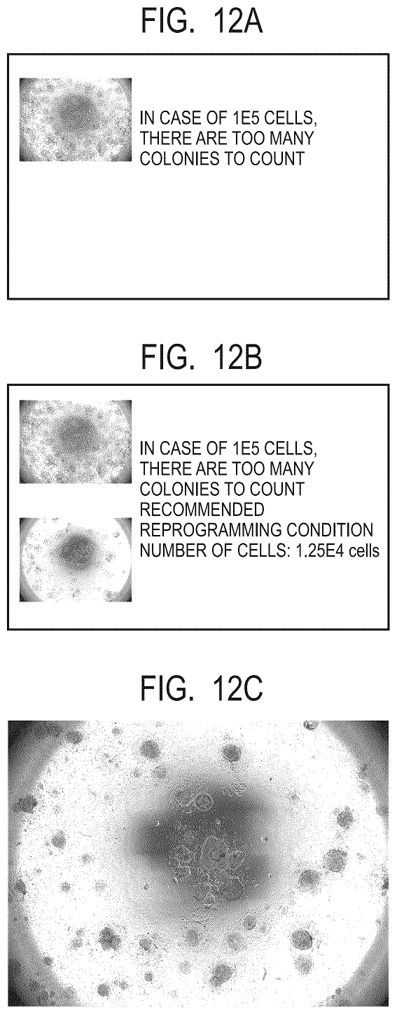

In this Example, when reprogramming was performed under the planned reprogramming condition, an estimation result that there were too many colonies to count, as shown in a result of the 1E5 cells/well of FIG. 11 was obtained. When such a state as to thus prevent counting of colonies due to overlaps thereamong is predicted, the estimation result is not required to be numerically displayed, and as shown in, for example, FIG. 12A, the display may be performed by indicating that there are too many colonies to count. The stored experimental data may also be used to present imagery of the bright-field images of the colonies after reprogramming.

Further, when the step of generating a recommended reprogramming condition was carried out in Step 3, a condition of performing reprogramming with the number of cells being 1.25E4 cells was presented. FIG. 12B shows an example of presentation of the estimation result and the recommended reprogramming condition that can be generated. Although a specific number of cells is presented in this case, it is also possible to display a dilution condition of the cells and an estimated amount in a case of using the current cell suspension. The stored experimental data may also be used to present imagery of the bright-field images of the colonies after reprogramming.

As described above, the use of the information processing method enables estimation of a result of the reprogramming of the subject blood cell and generation of a recommended reprogramming condition.

### Example 2

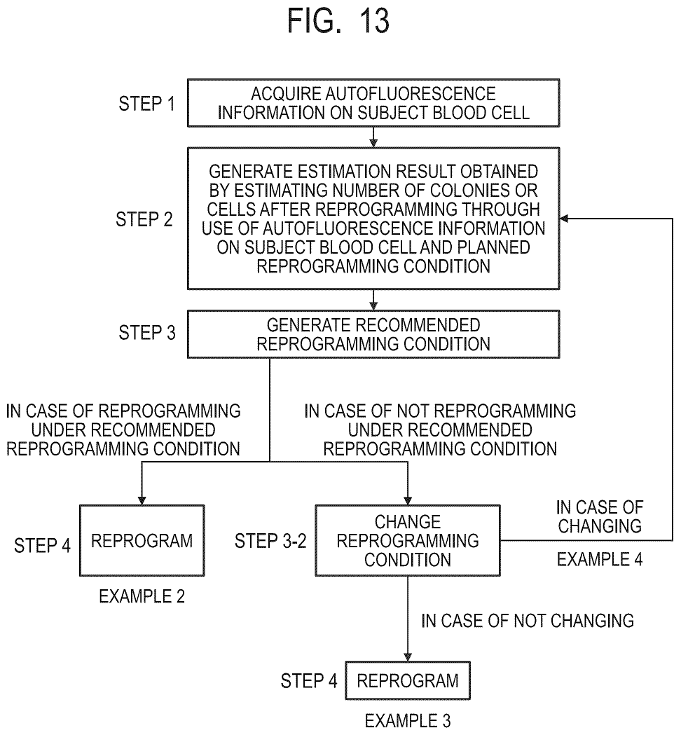

In FIG. 13, flows of Examples and corresponding Example numbers are illustrated.

In Example 2, in the same manner as in Example 1, the lot C was set as subject blood cells. In Example 1, as shown in FIG. 12B, the condition of performing reprogramming with the number of cells being 1.25E4 cells was presented as the recommended reprogramming condition. In view of this, the process proceeded to Step 4 of FIG. 13, and reprogramming was performed under the recommended reprogramming condition. The user can select whether or not to perform reprogramming under the recommended reprogramming condition. The selection can be performed through use of the input unit.

In Step 4, for example, the cell suspension is diluted so as to achieve 1.25E6 cells/mL, and reprogramming is carried out through use of 10 μL corresponding to 1.25E4 cells. The cell suspension corresponding to 1.25E4 cells may be dispensed and subjected to centrifugation and supernatant removal, and may be resuspended in 10 μL of the culture medium. As a result of carrying out reprogramming under the recommended reprogramming condition as described above, a result shown in FIG. 12C was obtained.

In this Example, reprogramming was performed with the number of cells having been reduced from the planned reprogramming condition. However, when an estimation result that the number of colonies that can be acquired is small is obtained in Step 2, a reprogramming condition in which the cell amount is increased is recommended in Step 3. In this case, since an increase in the volume of cell fluid affects the reprogramming efficiency, the necessary cells may be dispensed, the cells centrifuged to remove the supernatant, and the cells resuspended in a small solution, for example, 10 μL.

### Example 3

In this Example, in the same manner as in Examples 1 and 2, PBMCs obtained by pre-culturing commercially available PBMCs for 5 days were used, but a different lot thereof was used. The obtained cells are hereinafter referred to as “PBMC lot D.”

First, as a result of acquiring the autofluorescence information in Step 1 illustrated in FIG. 13, the ratio of the population having high brightness values of the blue component and the green component of the PBMC lot D was 0.05%.

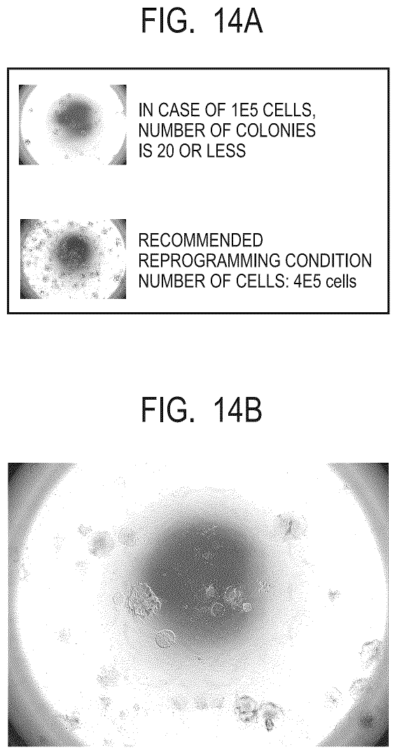

Subsequently, in Step 2, the autofluorescence information and the planned reprogramming condition were used to estimate the number of colonies after reprogramming and generate an estimation result. Information processing was carried out by using the same planned reprogramming condition as in Example 1. In this Example, it was estimated that, after execution of the planned reprogramming, the number of colonies was to be 20 or less as shown in FIG. 14A. In the generation of an estimation result and a recommended reprogramming condition, calculation thereof was performed from the experiment results shown in FIG. 11 in the same manner as in Example 1.

In Step 3, in the step of generating a recommended reprogramming condition, a condition of setting the number of cells to 4E5 cells was presented as shown in FIG. 14A. In Example 2, the process proceeded to Step 4 of obtaining a pluripotent stem cell by introducing a reprogramming factor through use of the reprogramming condition generated in Step 3. Meanwhile, this Example corresponds to a case in which reprogramming is not to be performed under the recommended reprogramming condition. That is, in this Example, it was impossible to prepare the number of cells indicated by the recommended reprogramming condition, and thus reprogramming was not performed under the reprogramming condition generated in Step 3. The user of the device can select, through the input unit, whether or not to perform reprogramming under the recommended reprogramming condition. When it is selected not to perform the reprogramming under the recommended reprogramming condition, the process proceeds to Step 3-2 after Step 3. In the case of this Example, it was impossible to proceed to Step 4 under the recommended reprogramming condition presented in Step 3, and thus it was selected not to perform the reprogramming under the recommended reprogramming condition.

Further, in Step 3-2, the user can select, with reference to the result of Step 3, whether to change the reprogramming condition or to proceed to Step 4 under a state in which the reprogramming condition is unchanged. The selection can be performed through the input unit attached to the device. In Example 3, due to inability to prepare the number of cells indicated by the recommended condition, it was selected not to change, the process proceeded to Step 4 with the unchanged condition, and reprogramming was carried out under a basic reprogramming condition. As a result, colonies as shown in FIG. 14B were obtained.

Even in a case in which, as in Example 2, the estimation result that there are too many colonies to count has been obtained and a condition indicating the reduced number of cells has been generated as the recommended reprogramming condition, it may be sometimes selected to perform reprogramming under an initial planned reprogramming condition without changing the reprogramming condition. In this case, growth of each of colonies is difficult to observe, but large numbers of colonies and pluripotent stem cells can be obtained. As described above, whether or not to carry out reprogramming under the recommended reprogramming condition can be selected by the user depending on a purpose. Further, the purpose may be set in advance in the device. The device may be provided with such a setting as to enable selection of the purpose such as to obtain colonies the number of which allows each of the colonies to be observed, to obtain as many pluripotent stem cells as possible, and the like.

### Example 4

In Example 3, it was impossible to prepare the number of cells indicated by the recommended reprogramming condition, and thus the reprogramming was performed under the initial planned reprogramming condition. In this Example, the recommended reprogramming condition was changed in an attempt to acquire as many pluripotent stem cells as possible.

The steps up to Step 3 were carried out by the same method as in Example 3 to generate a recommended reprogramming condition. As a result, the condition of setting the number of cells to 4E5 cells was presented as the recommended reprogramming condition. However, due to inability to prepare the recommended number of cells, in the same manner as in Example 3, it was selected not to perform reprogramming under the recommended reprogramming condition after Step 3 by the user of the device, and the process proceeded to Step 3-2. Subsequently, when it was selected to change the reprogramming condition in Step 3-2, the reprogramming condition was enabled to be changed, and the process returned to Step 2 of obtaining the estimation result again under the changed condition. In the case of this Example, due to inability to prepare a required number of cells, a case in which the culture period was set long, for example, 18 days was input is assumed. The user of the device can input a change of the reprogramming condition through use of the input unit. In Example 4, as a result of changing the number of culture days to 18 days, a result that a required amount of pluripotent stem cells can be obtained under the current condition in Step 2 and Step 3 was obtained, and thus the process proceeded to Step 4. As a result, a required amount of pluripotent stem cells was able to be obtained.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present disclosure, an information processing method for reprogramming of a blood cell comprising an analysis step utilizing autofluorescence is provided, thereby enabling stable pluripotent stem cell production. According to the present disclosure, the autofluorescence of a cell is utilized, and thus, staining with an antibody or the like is not required to be performed, and analysis can be performed non-invasively.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-041312, filed Mar. 15, 2023, which is hereby incorporated by reference herein in its entirety.

What is claimed is:

1. An information processing method for reprogramming of a blood cell, the information processing method comprising:
an autofluorescence information acquisition step of acquiring autofluorescence information on a subject blood cell; and
an analysis step of performing analysis based on the autofluorescence information,
wherein the analysis step comprises at least any one of:
an estimation step of generating an estimation result obtained by estimating a property after reprogramming based on a planned reprogramming condition; or
a recommended reprogramming condition generation step of generating a recommended reprogramming condition for the subject blood cell based on a desired property of the cell after reprogramming.

2. The information processing method for reprogramming of a blood cell according to claim 1, wherein the autofluorescence information comprises information about brightness of a blue component and a green component of autofluorescence.

3. The information processing method for reprogramming of a blood cell according to claim 2, wherein the blue component is fluorescence in a wavelength range of 350 nm or more and 500 nm or less, and the green component is fluorescence in a wavelength range of 500 nm or more and 600 nm or less.

4. The information processing method for reprogramming of a blood cell according to claim 1, wherein the analysis step comprises calculating a ratio of cells in which brightness values of a blue component and a green component are both high to the subject blood cells.

5. The information processing method for reprogramming of a blood cell according to claim 1, wherein the reprogramming condition comprises a condition relating to at least any one selected from the group consisting of: information on the number of cells; information on a reprogramming factor; information on a vector amount; information on the number of culture days; and information on a culture vessel.

6. The information processing method for reprogramming of a blood cell according to claim 1, wherein the property after reprogramming comprises information about the number of cells after reprogramming.

7. The information processing method for reprogramming of a blood cell according to claim 4, wherein the cells in which the brightness values of the blue component and the green component are both high in the subject blood cells are each a cell having a high stemness.

8. The information processing method for reprogramming of a blood cell according to claim 7, wherein the cell having a high stemness is a CD34-positive cell.

9. The information processing method for reprogramming of a blood cell according to claim 1, wherein the subject blood cell is a human peripheral blood mononuclear cell.

10. The information processing method for reprogramming of a blood cell according to claim 9, wherein the analysis step comprises referring to information about a correspondence relationship between the autofluorescence information and the reprogramming condition, and the property after reprogramming.

11. The information processing method for reprogramming of a blood cell according to claim 1, wherein the analysis step comprises performing the analysis based on the autofluorescence information and cell information comprising identification information on the subject blood cell.

12. The information processing method for reprogramming of a blood cell according to claim 11, wherein the analysis step comprises referring to information about a correspondence relationship between the cell information, the autofluorescence information, and the reprogramming condition, and the property after reprogramming.

13. The information processing method for reprogramming of a blood cell according to claim 1, wherein the analysis step comprises:
performing the analysis based on the autofluorescence information and cell information comprising identification information on the subject blood cell; and
further using a learning model obtained through use of training data in which the cell information, the autofluorescence information, and the reprogramming condition are set as input data and the property after reprogramming is set as output data.

14. The information processing method for reprogramming of a blood cell according to claim 1,
wherein the analysis step comprises a first recommended reprogramming condition generation step of generating a recommended first reprogramming condition for the subject blood cell based on a desired property 1 of the cell after reprogramming, and
wherein the analysis step further comprises, when an instruction is given by a user, a second recommended reprogramming condition generation step of generating a recommended second reprogramming condition for the subject blood cell based on a desired property 2 of the cell after reprogramming.

15. An information processing device for executing an information processing method for reprogramming of a blood cell, wherein the information processing method comprises:
an autofluorescence information acquisition step of acquiring autofluorescence information on a subject blood cell; and
an analysis step of performing analysis based on the autofluorescence information,
wherein the analysis step comprises at least any one of:
an estimation step of generating an estimation result obtained by estimating a property after reprogramming based on a planned reprogramming condition; or
a recommended reprogramming condition generation step of generating a recommended reprogramming condition for the subject blood cell based on a desired property of the cell after reprogramming.

16. An information processing program for causing an information processing device to execute an information processing method for reprogramming of a blood cell, wherein the information processing method comprises:
an autofluorescence information acquisition step of acquiring autofluorescence information on a subject blood cell; and
an analysis step of performing analysis based on the autofluorescence information,
wherein the analysis step comprises at least any one of:
an estimation step of generating an estimation result obtained by estimating a property after reprogramming based on a planned reprogramming condition; or
a recommended reprogramming condition generation step of generating a recommended reprogramming condition for the subject blood cell based on a desired property of the cell after reprogramming.

17. An electronic medium having stored thereon an information processing program for causing an information processing device to execute an information processing method for reprogramming of a blood cell, wherein the information processing method comprises:
an autofluorescence information acquisition step of acquiring autofluorescence information on a subject blood cell; and
an analysis step of performing analysis based on the autofluorescence information,
wherein the analysis step comprises at least any one of:
an estimation step of generating an estimation result obtained by estimating a property after reprogramming based on a planned reprogramming condition; or
a recommended reprogramming condition generation step of generating a recommended reprogramming condition for the subject blood cell based on a desired property of the cell after reprogramming.

18. A reprogramming device for a blood cell, the reprogramming device comprising:
an image pickup unit configured to acquire autofluorescence information on a subject blood cell;
an information processing system; and
a reprogramming factor introduction unit configured to introduce a reprogramming factor into the subject blood cell,
wherein the information processing system comprises:
an autofluorescence information acquisition unit configured to acquire the autofluorescence information from the image pickup unit; and
an analysis unit configured to perform analysis based on the autofluorescence information, and
wherein the analysis unit comprises at least any one of:
an estimation unit configured to generate an estimation result obtained by estimating a property after reprogramming based on a planned reprogramming condition; or
a recommended reprogramming condition generation unit configured to generate a recommended reprogramming condition for the subject blood cell based on a desired property of the cell after reprogramming.

19. The reprogramming device for a blood cell according to claim 18, wherein the analysis unit comprises the recommended reprogramming condition generation unit configured to generate the recommended reprogramming condition for the subject blood cell based on the desired property of the cell after reprogramming, and
wherein the reprogramming factor introduction unit is configured to introduce the reprogramming factor into the subject blood cell in accordance with the recommended reprogramming condition.

20. A method of manufacturing a pluripotent stem cell, the method comprising:
an autofluorescence information acquisition step of acquiring autofluorescence information on a subject blood cell;
an analysis step of performing analysis based on the autofluorescence information; and
a step of reprogramming the subject blood cell,
wherein the analysis step comprises at least any one of:
an estimation step of generating an estimation result obtained by estimating a property after reprogramming based on a planned reprogramming condition; or
a recommended reprogramming condition generation step of generating a recommended reprogramming condition for the subject blood cell based on a desired property of the cell after reprogramming.

21. The method of manufacturing a pluripotent stem cell according to claim 20,
wherein the analysis step comprises the recommended reprogramming condition generation step of generating the recommended reprogramming condition for the subject blood cell based on the desired property of the cell after reprogramming, and
wherein the method further comprises reprogramming the subject blood cell in accordance with the recommended reprogramming condition.

22. The method of manufacturing a pluripotent stem cell according to claim 20,
wherein the analysis step comprises:
the estimation step of generating the estimation result obtained by estimating the property after reprogramming based on the planned reprogramming condition; and
the recommended reprogramming condition generation step of generating the recommended reprogramming condition for the subject blood cell based on the desired property of the cell after reprogramming, and
wherein the method further comprises reprogramming the subject blood cell in accordance with the recommended reprogramming condition.

23. The method of manufacturing a pluripotent stem cell according to claim 20,
wherein the analysis step comprises:
a first recommended reprogramming condition generation step of generating a recommended first reprogramming condition for the subject blood cell based on a desired property 1 of the cell after reprogramming; and
a second recommended reprogramming condition generation step of generating a recommended second reprogramming condition for the subject blood cell based on a desired property 2 of the cell after reprogramming, and
wherein the method further comprises reprogramming the subject blood cell in accordance with the recommended second reprogramming condition.
